(19) United States
(12) Patent Application Publication
(10) Pub. No.: US 2019/0161785 A1
(43) Pub. Date: May 30, 2019

(54) Systems, Devices and Methods for Sequential Analysis of Complex Matrix Samples for High Confidence Bacterial Detection and Drug Susceptibility Prediction Using a Flow Cytometer

(71) Applicant: Aperture Bio, LLC, Williston, VT (US)

(72) Inventors: Matthew D. Gombrich, Underhill, VT (US); Shawn Ramsaroop, St. Louis, MO (US); Margaret Bozzuti, Richmond, VT (US)

(21) Appl. No.: 16/096,549

(22) PCT Filed: Apr. 25, 2017
(86) PCT No.: PCT/US2017/029492

§ 371 Date: Oct. 25, 2018

Related U.S. Application Data
(60) Provisional application No. 62/470,595, filed on Mar. 13, 2017.
(60) Provisional application No. 62/327,007, filed on Apr. 25, 2016.

(51) Int. Cl. C12Q 1/06; G01N 15/10; C12Q 1/18

(52) U.S. Cl. CPC C12Q 1/06; G01N 15/1012; G01N 2015/1486; G01N 2015/1006; C12Q 1/18

(57) ABSTRACT

Method for testing a sample of body fluid for the presence of bacteria and comprising the steps of adjusting the dilution of the sample to a predetermined concentration, of dividing the diluted sample into two batches defining a baseline-batch and a control-batch, of testing the baseline-batch at a time T0 with the flow cytometer to obtain enumerative baseline bacterial values, of culturing the control-batch in growth media between times T0 and T1, of testing the control-batch at time T1 with the flow cytometer to obtain enumerative control bacterial values, and of comparing the control values to the baseline values to determine a bacteria growth-ratio.

### RELATED APPLICATION DATA

This application claims the benefit of priority of U.S. Provisional Patent Application Ser. No. 62/327,007, filed Apr. 25, 2016, and titled “Analytical Method For Enumerative Compensation Using A Flow Cytometer”, and U.S. Provisional Patent Application Ser. No. 62/470,595, filed Mar. 13, 2017, and titled “Flow Cytometer Systems Including Automated Fluid Handling Systems and Methods of Using the Same for Quantifying the Effectiveness of Antimicrobial Agents”, each of which is incorporated by reference herein in its entirety.

### FIELD OF THE INVENTION

The present invention generally relates to the field of sequential analysis of complex matrix samples for high confidence bacterial detection in bodily fluid samples. In particular, the present invention is directed to systems, devices and methods for sequential analysis of complex matrix samples for high confidence bacterial detection and drug susceptibility prediction using a flow cytometer.

### BACKGROUND

The use of flow cytometer based systems for detecting and enumerating contaminants in liquid samples is well known. Additionally, flow cytometry has been utilized for detection of bacteria in fluid samples. Numerous patent and non-patent publications claim to present flow cytometry systems and methods capable of “rapid” detection or diagnosis of bacteria or infection in fluid samples. Examples of such publications include Sakai et al., U.S. Pat. No. 7,645,594, Method of Preparing Assay Sample For Discriminating Bacteria by Flow Cytometer; Tanaka et al. U.S. Pat. No. 8,333,926, Apparatus for Analyzing Particles in Urine and Method Thereof; Mansour et al., U.S. Pat. No. 4,622,298, Detection and Quantitation of Microorganisms, Leukocytes and Squamous Epithelial Cells in Urine; Groner et al., U.S. Pat. No. 5,858,697, Method of Rapid Diagnosis of Urinary Tract Infections; Czarnek, U.S. Pat. No. 7,468,789, Flow Cytometer for Rapid Bacteria Detection; Kawashima, EU 1466985, Methods for Measuring Bacteria, Bacteria Measuring Apparatus, and Storage Media for Storing Computer-Executable Programs for Analyzing Bacteria; Nuding et al., Detection, Identification and Susceptibility Testing of Bacteria by Flow Cytometry, J. Bacteriol Parasitol 2013, S:5; Walberg et al., Rapid Flow Cytometric Assessment of Mecillinam and Ampicillin Bacterial Susceptibility, Journal of Antimicrobial Chemotherapy (1996) 37, 1063-1075; and Durodie et al., Rapid Detection of Antimicrobial Activity Using Flow Cytometry, Cytometry 21:374-377 (1995).

While such systems and methods might be characterized as “rapid” in comparison to prior bench-based incubation methods, current flow cytometer-based systems and methodologies are limited in their capabilities due to inaccuracies and other drawbacks arising from a variety of factors. There thus remains a need in the art for systems, devices and methods capable of rapid detection, diagnosis and testing with greater accuracy and reduced false positive results.

### SUMMARY OF THE DISCLOSURE

In one implementation, the present disclosure is directed to a method of using a flow cytometer in an automated fluid handling system for testing a clinical sample of a body fluid for the presence of bacteria, and optionally determining sample response to at least one antibiotic. The method includes distributing a portion of the sample to at least a first test well using the automated fluid handling system; testing the sample portion from the first well with a flow cytometer to determine a total bacteria count; adjusting dilution of the sample with growth media to a predetermined concentration based on the total bacteria count; dividing the dilution-adjusted sample into at least wells including a time 0 baseline (T₀ baseline) well and a time 1 control (T₁ control) well; testing the sample in the T₀ baseline well at time T₀ with the flow cytometer to obtain T₀ enumerative baseline bacterial values relating to measured characteristics of the sample in the T₀ well; culturing the sample in the T₁ control well from time 0 to time 1; testing the T₁ control batch at time 1 with the flow cytometer to obtain T₁ enumerative control bacterial values relating to measured characteristics of the T₁ sample; and comparing the T₁ control values to the T₀ baseline values to determine a growth ratio of samples containing bacteria.

In another implementation, the present disclosure is directed to a method of using a flow cytometer for testing a sample of a body fluid for the presence of bacteria, and optionally determining sample response to at least one antibiotic. The method includes adjusting dilution of the sample to a predetermined concentration; dividing the diluted sample into at least two batches including a time 0 baseline (T₀ baseline) batch and a time 1 control (T₁ control) batch; testing the T₀ baseline batch at time T₀ with the flow cytometer to obtain T₀ enumerative baseline bacterial values relating to measured characteristics of the T₀ batch; culturing the T₁ control batch in growth media from time 0 to time 1; testing the T₁ control batch at time 1 with the flow cytometer to obtain T₁ enumerative control bacterial values relating to measured characteristics of the T₁ sample; comparing the T₁ control values to the T₀ baseline values to determine a growth ratio of samples containing bacteria.

In yet another implementation, the present disclosure is directed to a method of compensating for inaccuracies in flow cytometer enumeration of particles of interest in fluid samples. The method includes including a known concentration of a test-enumerative compensator (TEC) particles in the sample to be enumerated, said TEC particles having known flow cytometric scatter and fluorescence characteristics; enumerating the TEC particles with the sample enumeration by the flow cytometer; determining a compensator factor based on the enumerated TEC particle value as compared to the known TEC particle concentration in the sample tested; and adjusting the sample test enumeration value by said compensator factor.

In still another implementation, the present disclosure is directed to a system for automated testing a sample of a body fluid for the presence of bacteria, and optionally determining sample response to at least one antibiotic. The system includes fluid handling device including an automated pipetting system for distributing fluid samples among wells of a well plate; incubator configured to culture samples in well plates received from the fluid handling device; plate transport device configured to deliver well plates containing samples to the incubator from the fluid handling device and return well plates from the incubator to the fluid handling device; flow cytometer configured to enumerate cell counts in samples provided by the fluid handling system; processor and memory, the processor configured execute instructions stored in the memory to control the system in accordance with said instructions, wherein the stored instructions cause the system to distribute a portion of the sample to at least a first test well; enumerate the sample portion from the at least first test well to determine a total bacteria count; adjust dilution of the sample with growth media to a predetermined concentration based on the total bacteria count; divide the dilution-adjusted sample into at least wells including a time 0 baseline (T₀ baseline) well and a time 1 control (T₁ control) well; enumerate the sample in the T0 baseline well at time T₀ to obtain T₀ enumerative baseline bacterial values relating to measured characteristics of the sample in the T₀ well; deliver the sample to the incubator; culture the sample in the T₁ control well from time 0 to time 1; return the sample to the fluid handling device after culturing; enumerate the T₁ control batch at time 1 with the flow cytometer to obtain T₁ enumerative control bacterial values relating to measured characteristics of the T₁ sample; and compare the T₁ control values to the T₀ baseline values to determine a growth ratio of samples containing bacteria; and a graphical user interface communicating with at least the processor allowing user interaction with the system.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a depiction of the hardware employed in a system according to one embodiment disclosed herein.

FIG. 2 is a block diagram schematically depicting functional units of a system according to an embodiment disclosed herein.

FIG. 3 is a high-level flow diagram depicting a method according to an embodiment disclosed herein.

FIG. 4 is a schematic depiction of a fluid handling system and multi-well cassette according to one embodiment disclosed herein.

FIG. 5 is a schematic depiction of an alternative multi-well cassette.

FIG. 6 is a schematic depiction of a further alternative multi-well cassette.

FIG. 7 is a flow diagram depicting a method according to an embodiment disclosed herein including identifying bacteria and testing for resistance/susceptibility to antibiotics.

FIG. 8 is a flow diagram depicting an alternative method according to another embodiment disclosed herein.

FIGS. 9A, 9B and 9C are cytograms representing a eukaryotic screening enumeration of a clinical specimen depicted as scatter plots on exemplary regions of interest (ROI), wherein the grey-shaded areas represent predetermined ROIs (here, as well as in all other cytograms presented herein).

FIGS. 10A and 10B are cytograms representing bacterial screening enumeration of a clinical specimen that has been diluted and depicted as scatter plots on exemplary regions of interest (ROI).

FIGS. 11A and 11B are cytograms representing bacterial enumeration of another clinical specimen representing a T₀ control sample.

FIGS. 12A and 12B are cytograms representing bacterial enumeration of the clinical specimen represented in FIGS. 11A and 11B clinical specimen, but enumerated to determine the T₁ growth rate.

FIGS. 13A and 13B are cytograms representing bacterial enumeration of another clinical specimen that has been diluted and provided with test-enumerative compensator particles, depicted as scatter plots on exemplary regions of interest (ROI).

FIGS. 14A and 14B are cytograms representing bacterial enumeration of a further clinical specimen that has been diluted and provided with test-enumerative compensator particles, depicted as scatter plots on exemplary regions of interest (ROI).

FIG. 15 is a block diagram depicting components of an alternative control system according to the present disclosure.

### DETAILED DESCRIPTION

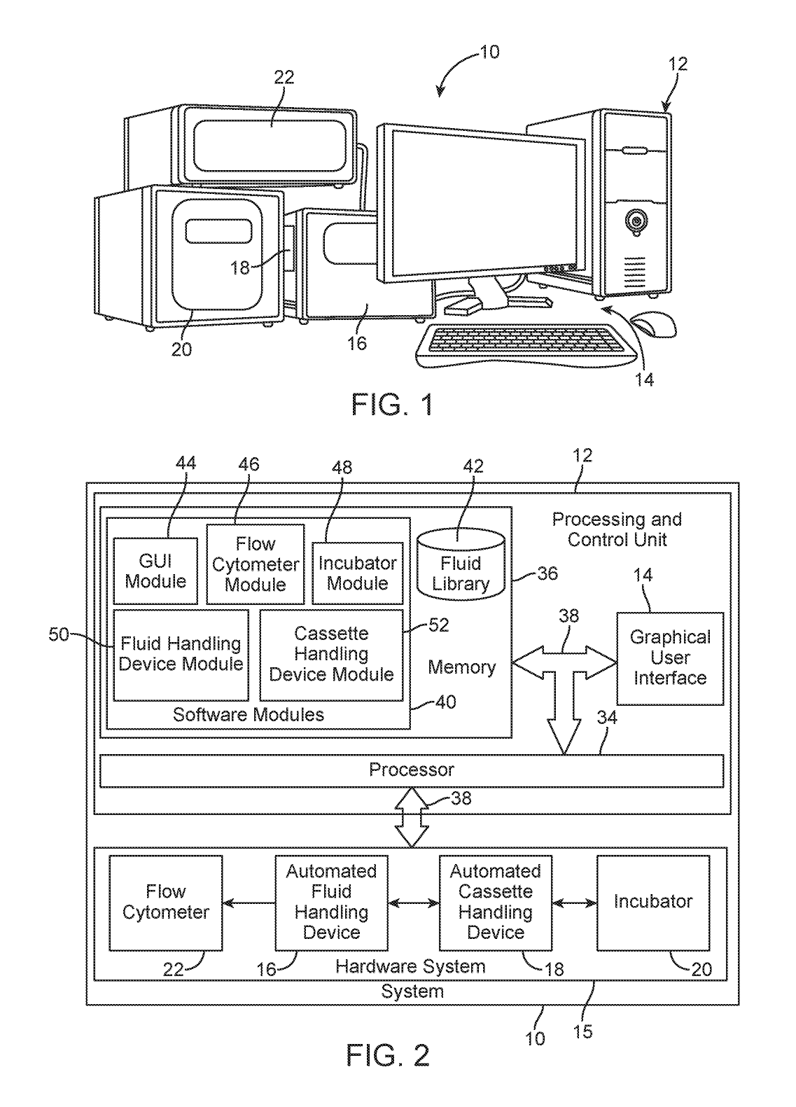

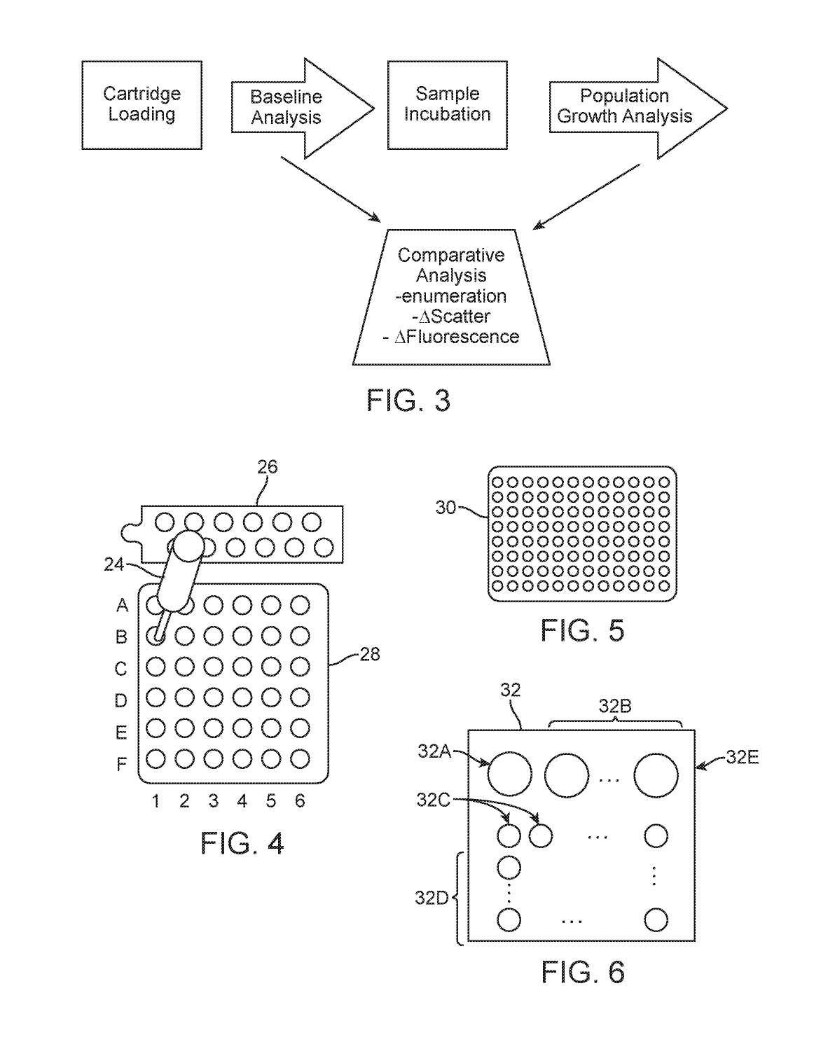

The present disclosure includes processes for loading a clinical sample in a testing cassette well and using quantitative results from the initial sample testing to inform subsequent sample processing by an automated fluid handling flow cytometer system for subsequent analysis, such as antibiotic effectiveness. FIGS. 1-6 and 15 illustrate exemplary automated fluid handling flow cytometer systems, including a flow cytometer for analyzing a fluid such as urine, blood, or cerebral spinal fluid, a fluid handling system that, as described below, may be configured to provide specific concentrations of fluid mixtures at specific times to the flow cytometer for testing, a wash system for washing fluid lines in the fluid handling system between samples, a cassette transporter for transporting a fluid cassette from an incubator to the fluid handling system, and an incubator for incubating a plurality of cassettes. The system may also include a variety of software programs for operating the system.

Embodiments of the present invention are directed to systems, methods and devices for improving bacterial discrimination in complex matrices by using short-course growth parameters and comparing the expansion of bacterial populations to an original control test assessed prior to growth. Embodiments described herein provide advantages over prior techniques and systems by providing quantitative methods for more accurately distinguishing live cells from dead cells within a body fluid sample and, in the case of bacteria, distinguishing between pathogenic bacteria of interest and contaminant bacteria that may be living and present in a sample, but not of clinical interest. In addition, the short-course growth protocol will include the ample being divided and incubated in the presence of multiple classes of antibiotics, providing for expedited and accurate measurement of sample response to specific antibiotics sufficient to provide a predictive profile of the anticipated resistance or susceptibility of the bacteria to the tested antibiotic (hereinafter referred to as “antibiotic predictive profile” or “APP”). In some instances the measured sample responses also may be useful in connection with other clinical tests, such as antibiotic susceptibility testing (AST). APP testing according to methods disclosed herein can provide a rapid antibiotic susceptibility/resistance profile of the bacterial contaminant if present. The assessment of the original and growth expansion is enumerated with appropriate assay reagents and a flow cytometer using an integrated software analysis program. This approach improves upon current methods by more accurately and rapidly assessing bacterial levels that may be indicative of infection and bacterial growth characteristics using nucleic acid intercalating dyes and assessment of bacterial scatter characteristics to enumerate bacteria.

A second embodiment relates to the sample cassette where a sample suspected of containing bacteria is loaded and the sample is divided equally among a number of wells containing growth media and, in some cases, antibiotics, for analysis and incubation at appropriate temperature to promote bacterial division. Flow cytometric software enables the user to assess changes in scatter characteristics and fluorescence characteristics of the bacterial population due to the effects of an antimicrobial agent. Changes in population enumeration, population statistics such as, but not limited to range and coefficient of variation, and changes in scatter characteristics can be used to provide confidence intervals to the user regarding bacterial isolate vulnerability to classes of antimicrobials.

In one embodiment of the invention, flow cytometric software enables a user to assess changes in population expansion due to bacterial division at multiple times with the aim of reducing enumeration of non-bacterial events and differentiating pathogenic bacteria from non-pathogenic contaminant bacteria. Another embodiment provides an automated system wherein a sample cassette where a sample suspected of containing bacteria is loaded and the sample is divided equally among a number of wells containing growth media and, in some cases, antibiotics, for analysis and incubation at appropriate temperature to promote bacterial division. Flow cytometric software enables the user to assess changes in scatter characteristics and fluorescence characteristics of the bacterial population due to the effects of an antimicrobial agent. Changes in population enumeration, population statistics such as, but not limited to range and coefficient of variation, and changes in scatter characteristics can be used to provide confidence intervals to the user regarding bacterial isolate vulnerability to classes of antimicrobials.

An exemplary embodiment of systems described herein is schematically depicted in FIGS. 1 and 2. At a high level, system 10 may include processing and control unit 12 with a graphical user interface (GUI) 14 to allow a user to control operation of system hardware components. Hardware system 15 may include hardware components such as fluid handling system 16, automated cassette handling system 18, incubator 20 and flow cytometer 22. Fluid handling system 16 may include, for example, automated pipetting system 24 (shown in FIG. 4), as well as one or more cassette handling robots and microplate washers. Except as otherwise noted herein, these hardware components may be selected from among commercially available devices for performing the described functions and configured by persons of ordinary skill based on knowledge in the art in light of the teachings presented herein.

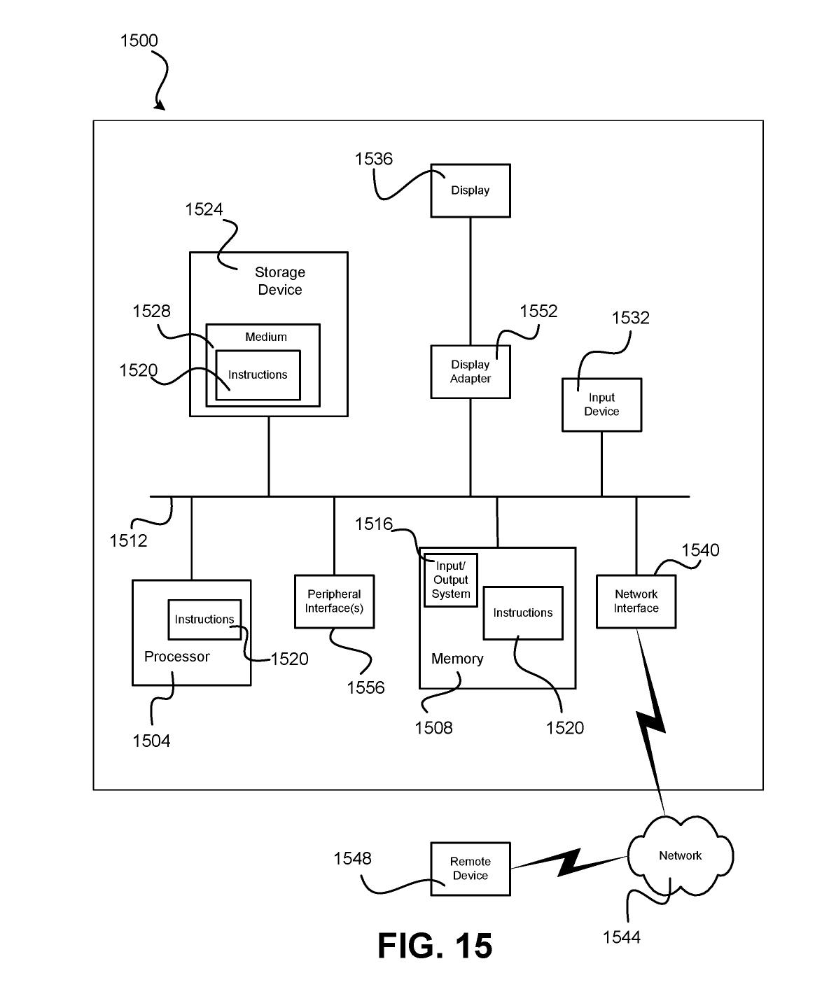

Processing and control unit 12, as illustrated in FIG. 2 may comprises processor 34 and memory 36. The memory and processor communicate with GUI 14 and hardware system 15 through appropriate application programming interfaces (API) and communication buses 38. Configurations with respect to processor communication and control are described in more detail below with respect to FIG. 15 Components of memory 36 may include software modules 40 configured specifically to control and operate the connected hardware components and fluid library 42. Exemplary software modules may comprise GUI module 44, flow cytometer module 46, incubator module 48, fluid handling device module 50, and cassette handling device module 52. Fluid library 42 is populated with fluid and bacteria specific information used for analyzing the particular type of fluid under analysis, such as, but not limited to, urine, spinal fluid and blood. For example, flow cytometer software module 46 may access pre-defined regions of interest (ROIs), scatter values and fluorescence values etc. stored in the fluid library for detecting various species of bacteria in various fluids being tested. Detections in the ROI possessing characteristics of target events, such as scatter values and fluorescence values, as determined by gating strategies and/or computational analysis executed by the flow cytometer software may be used to determine concentration of particles, cells or bacteria of interest in the sample. Also, described in more detail below, multiple ROIs for particular fluid types may be stored for use at different points of an analysis.

As will be appreciated from descriptions of various embodiments presented herein, one exemplary embodiment may comprise a kit for general bacterial staining or other analysis, including a multi-well cassette containing growth media and antibiotics in designated wells. The growth media may be provided in dried, freeze dried, or other preserved from, which may be activated, such as by hydration, in an initial processing step within or prior to placement in a fluid handling system. As used herein, multi-well cassette may refer to any cassette, plate or well structure adapted for automated assaying and fluid handling. One example of such a cassette may be a 6×6 Eppendorf tube rack 28 (and associated tubes), as shown in FIG. 4, in which wells are formed by individual, removable tubes. Other examples of multi-well cassettes, as that term is used herein, may include a conventional microwell plates, such as ninety-six well plate 30, shown in FIG. 5. Other alternative or custom multi-well cassettes 32 (FIG. 6), with varying size wells for particular applications may be devised. With reference again to FIG. 4, automated pipetting system 24 of fluid handling system 16 may be used to distribute fluids as between wells of the multi-well cassette.

In one exemplary embodiment, a single volume of sample is loaded in the cassette and distributed into all sample wells equally. This includes a T₀ control well, T₁ control well, and all wells containing antibiotics. The sample would be loaded onto the automated flow cytometer and the sample ID assigned to the cassette. T₀ control values would be determined for any population assigned to the bacterial ROI, including enumeration, mean fluorescence, CV, range of the population, and calculation of “distance” from the compensation beads in the sample. This data would be stored and used as a reference for subsequent analysis after sample incubation at T₁.

During use, a fluid sample is divided among the wells of the cassette. The sample is treated with a staining reagent that stains at least live bacteria, and in some cases, also stains dead cells to differentiate between live and injured/dead bacteria cells. One or more antimicrobial agents can be selected and added to various wells to test the efficacy of the antimicrobial agents for treating any bacteria that is present. There are at least two control wells (T₀ and T₁ wells that contain growth media but no antimicrobial) and in some cases at least one additional well for testing an antimicrobial agent.

The T₀ control well sample is analyzed with a flow cytometer that includes a bacteria library that defines a region of interest (ROI) tailored to bacteria. The T₀ sample is tested at time zero to obtain baseline T₀ control values for any population assigned to the bacterial region of interest (ROI), including enumeration, mean fluorescence, CV, range of the population, and if fluorescent compensation beads are used, a calculation of a distance from the compensation beads in the sample. This data is stored and used as a reference for subsequent analysis after sample incubation at T₁.

Subsequent volumes of the sample are incubated at a pre-determined time and temperature (e.g. 37 C) and analyzed by the system at T₁, and perhaps beyond. Comparative analysis to the initial T₀ sample would ensue to determine population expansion in the pre-determined bacteria-specific ROI.

If the T₁ control sample contains bacteria, it will have population expansion in the ROI relative to the initial T₀ test results. Such expansion will help ensure detected events are in fact bacteria and not spurious data, such as noise or non-bacteria particles. In other words, samples not containing bacteria should have no change in ROI population such that any detected events in the bacteria ROI are most likely cell debris or noise.

Multiple T₁ tests can be run, each T₁ sample having been incubated with one or more clinically relevant chemicals and/or antibiotics. Susceptible bacterial strains will show a decrease in live cell event numbers in the bacteria ROI relative to the T₁ control as well as potential changes in scatter characteristics assessed with the flow cytometer. Resistant bacterial strains would show less or no decrease in bacterial number relative to the T₁ control as well as no changes in scatter characteristics.

In addition to high-confidence bacterial identification, this technique can be applied to the identification of co-infections (i.e., the sample contains more than one species of bacteria) when used in conjunction with antibiotic susceptibility testing. With sequential analysis of samples incubated with specific antibiotics, the ability to look at the live cell ROI for multiple sub-populations, e.g., two populations that respond differently to the antibiotic in the sample, becomes a reality.

The use of image analysis software allows for tracking of event density within the live cell ROI. For example, while two species of bacteria may generally have the same scatter and fluorescence characteristics as compared to other particles in the same sample such as cell debris and leukocytes, the different bacteria species may have slightly different parameters, e.g., scatter. When testing subsequent T₁, T₂, etc. samples treated with an antibiotic, in cases where the antibiotic has more efficacy for one species than another, the characteristics of the events in the live cell ROI may begin to asymmetrically shift, suggesting two or more populations.

Where antibiotic effect shows clear differences in population change with the region, a confidence interval can be ascribed to the likelihood of more than one species of bacteria being present in the sample.

As an example, the present invention relates to determining bacteremia in clinical blood samples by allowing a clinical laboratory to load a pre-determined volume of blood into multiple testing wells, all of which contain growth media and some of which contain antibiotics at clinically relevant concentrations. Automated software and hardware would analyze the sample at time zero (T₀) using appropriate dyes targeting bacteria that discriminate live cells from injured or dead cells. Values in samples containing positive populations would be determined and would include population enumeration, population mean fluorescence, population fluorescence CV, and population range. This T₀ template would be saved by the system and used as a reference result for all future testing. Subsequent volumes of the sample would be incubated at a pre-determined time and temperature (i.e. 37 C) and analyzed by the system at T₁, and perhaps beyond. Comparative analysis to the initial T₀ sample would ensue to determine population expansion in pre-determined regions of interest (ROI) that correlate significantly to bacteria. A sample containing bacterial contamination will have population expansion in the ROI relative to the initial test results. Samples not containing bacteria should have no change in ROI population that are most likely cell debris or noise. As well, multiple T₁ tests will take place where the sample has been incubated with clinically relevant chemicals and antibiotics. Susceptible bacterial strains will show a decrease in event numbers in the ROI relative to the T₁ control as well as potential changes in scatter characteristics assessed with the flow cytometer. Resistant bacterial strains show no decrease in bacterial number relative to the T₁ control as well as no changes in scatter characteristics.

In one exemplary embodiment, an automated method for analyzing a sample for the presence of bacteria and for determining the bacteria's antibiotic susceptibility includes depositing the sample in a multi-well cassette configured for use in fluid handling system 16 of overall system 10. The cassette may also have a predetermined volume of growth media, e.g., 1 ml, in one or more media wells (see, e.g. FIGS. 4-6). In one example, Mueller Hinton Broth may be used as the growth media. The cassette may also have a plurality of antibiotic wells containing predetermined amounts of antibiotics. Fluid handling system 16 may have one or more wells, such as in reagent rack 26, with dyes or other staining agents for staining the fluid sample for analysis by flow cytometer 22. In one example a live/dead cell dye may be used. The cassette with the fluid sample deposited in the sample well may be loaded into the fluid handling system by hand, or may be loaded with a plurality of other cassettes into the incubator and automatically loaded from the incubator into the fluid handling system by automated cassette handling system 18.

Once in fluid handling system 16, the fluid handling system may utilize automated pipetting system or other suitable probe 24 to remove, e.g., aspirate, a predetermined amount of live/dead cell dye from a dye well stored in the fluid handling system and deposit the dye in the sample well containing the fluid sample. The fluid handling system may also be configured to deposit a predetermined amount of standardized fluorescent beads into the fluid sample which can be used to verify the accuracy of the flow cytometer measurements as discussed in further detail below. In other examples, dyes and beads may be manually added to the sample well.

Fluid handling system 16 may also be programmed to perform a mixing process to adequately mix the dye with the sample. The cassette may then be incubated in a temperature-controlled incubation chamber of the fluid handling system for a predetermined period of time to enable the dye to react with and stain the bacteria. Once the dye incubation time has passed, the fluid handling system may automatically transport a predetermined amount of the fluid sample from the sample well to the flow cytometer for an initial measurement.

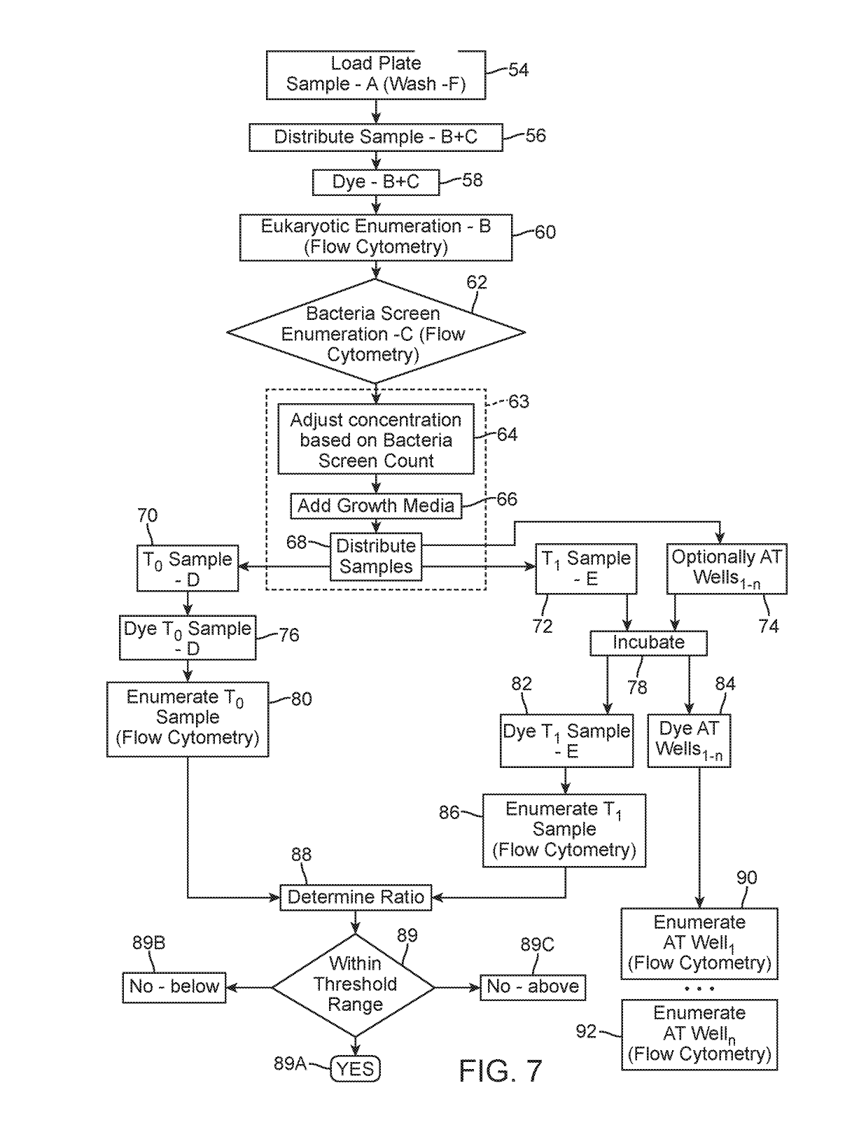

In one embodiment, with reference to FIG. 7, an exemplary automated method may begin with loading a sample into a multi-well cassette (step 54). Automated pipetting system 24 (or other suitable fluid delivery device) of fluid handling system 16 distributes the sample in appropriate quantities from well A to wells B and C (step 56). Note that for clarity of description, this exemplary embodiment is described with respect to a single “column” of wells, i.e. A1-F1 in FIG. 4. Persons of ordinary skill will appreciate that any number of columns and wells may be employed for simultaneous analysis of multiple samples.

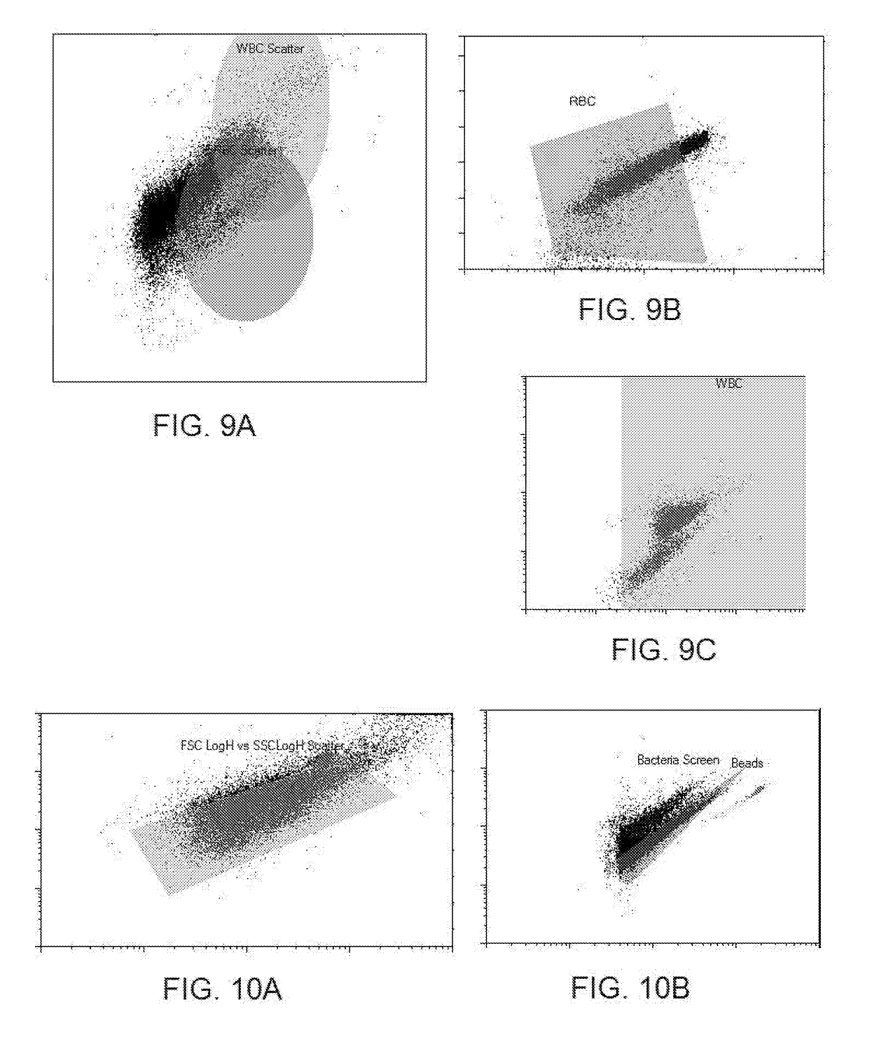

At step 58, automated pipetting system 24 obtains appropriate cellular stain (e.g., propidium iodide or thyzol orange) from designated wells of reagent rack 26 and the samples in wells B and C are stained. Then the fluid handling system delivers the contents of well B to flow cytometer 22 for eukaryotic enumeration at step 60. Exemplary results of enumeration step 60 are illustrated in the FIG. 9A scatter plot and fluorescence plots in FIGS. 9B and 9C. The ROIs in FIG. 9A provide gates for red and white blood cell counts. As is well understood in the flow cytometer art, events falling within the respective ROIs (including events falling within both ROIs) are further analyzed based on fluorescence intensity as shown in FIGS. 9B (red blood cell count, “RBC”) and 9C (white blood cell count, “WBC”). Again, events falling within the identified ROIs represent the cell counts. Typically WBC is reported, but depending on clinical requirements, the WBC and/or RBC may be reported.

In one embodiment, next, at step 62, contents of sample well C are delivered by fluid handling system 16 to flow cytometer 22 for bacteria screen enumeration. Scatter plot gating and fluorescence intensity analysis, as illustrated in FIGS. 10A and 10B, respectively, are again used to determine a bacteria count corresponding to events falling within the ROI of FIG. 10B. In some embodiments, dye applied to well C at step 58 may comprise at least two different dyes, for example one dye that permeates only dead cells and another that permeates all cells. Using distinct dye types in this manner allows for discrimination between live and dead cells based on the different fluorescence characteristics of the different dyes. The bacteria screen count of step 62, which may exclude dead cells depending on techniques employed, is compared against predetermined threshold values to assess whether continued analysis of the sample is warranted. For example, current clinical standards relative to assessment of urinary tract infections indicate thresholds of 10⁴/ml or 10⁵/ml depending on factors such as clinical status of the patient. Other threshold values may be applied as appropriate for analysis of other clinical indications or other clinical situations. Persons of ordinary skill will appreciate that in other embodiments the enumerations of steps 60 and 62 may be performed in reverse order, or, alternatively, to the extent not excluded by hardware or system limitations, simultaneously performed.

It should be noted that while the bacteria screen step 62 may be conducted to largely eliminate dead cells from the cell count based on use of fluorescence discriminating dyes, cell count at this stage still may include all types of live cells, both live cells of interest and live cells that are not of interest that may thus be considered as contaminant cells. For example, in assessment of urinary tract infections, a primary pathogenic bacteria of interest is e coli. However, a typical human urine sample may also include many different species of non-pathogenic flora. These non-pathogenic flora may be considered as contaminants with respect to accurate clinical analysis of pathogens.

Based on bacterial count determined in the preceding steps, in step 63 sample concentration is adjusted and samples distributed to further wells as needed for the analysis to be performed. Depending on hardware capabilities, step 63 may comprise individual steps as follows, which may be performed in a single operation or sequentially. Adjustment of sample concentration 64 can be accomplished by addition of appropriate amounts of growth media 66 when samples are further distributed 68 by automated pipetting system 24. Sample distribution 68 will include at least distribution of the T₀ sample to well D 70 and the T₁ sample to well E 72. Optionally further samples may be distributed to antibiotic testing (AT) wells 74 if APP or other antibiotic testing is to be included in the analysis. In one embodiment, adjustment step 64 is accomplished by depositing a properly diluted sample in an initial well (e.g., well D) and then distributing an amount of the sample from the initial well to all other wells to be employed.

As is known in the art, testing of bacteria for antibiotic resistance or susceptibility typically requires a bacterial concentration in the range of approximately 5×10⁴ to approximately 5×10⁵ bacteria/ml. However, depending on the sensitivity and accuracy of the instrumentation employed (for example some flow cytometer systems are more sensitive than others), lower concentrations may be employed. Thus, methods of the present disclosure may be employed with concentrations as low as in the range of 1×103 bacteria/ml. For example, instrument sensitivity may indicate a concentration in the range of approximately 1×10⁴ bacteria/ml to approximately 5×10⁴ bacteria/ml, or other instrumentation may employ a concentration in the range of approximately 1×10³ bacteria/ml to approximately 5×10³ bacteria/ml.

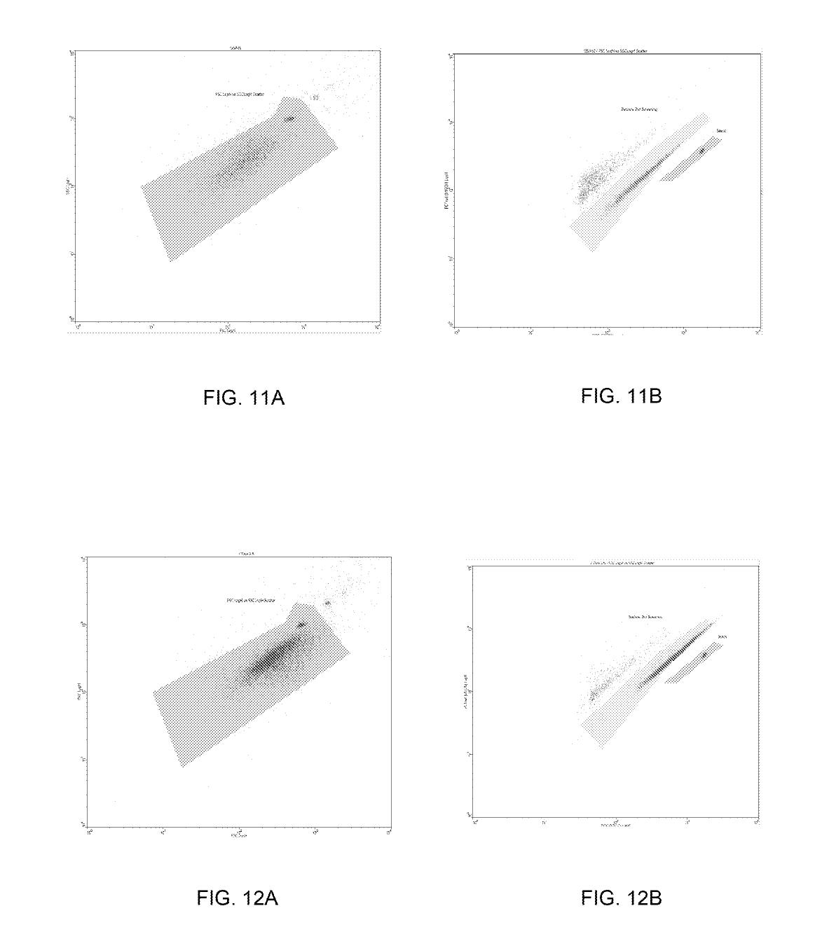

In one embodiment, sample concentration is adjusted for in bands or ranges. For example, to simplify processing without negatively impacting results, three bands may be used: A) 50-4999/μl, B) 5000-24999/μl, and C) 25000-40000/μl. Other bands or numbers of bands may be derived by persons of ordinary skill based on parameters such as desired speed and accuracy, instrument sensitivity and clinical objectives. A standard dilution may be employed for each concentration band to adjust the concentration to the desired range when depositing samples at step 63 as explained above. Subsequently, at step 76 the stain is added to sample T₀ and the T₀ sample is then enumerated at step 80 by flow cytometer 22 after delivery via fluid handling system 16. The T₀ sample serves as a control, against which growth rate is subsequently measured. FIGS. 11A and 11B illustrate results of this enumeration.

After sample T₀ is directed to enumeration at step 80, the multi-well cassette containing sample T₁ and any desired AT samples is delivered to incubator 20 by automated cassette handling system 18 and incubated at step 78. AT wells may be prefilled with specific antibiotics against which testing is to be run or may be separately filled from appropriate source wells by the fluid handling system. Incubation time will depend on the nature of the cells to be studied. For example, with respect to cells of interest, such as urogenital flora, incubation time may be in the range of about 2.5 hours, or typically less than about 3 hours, but more than 2 hours. After incubation, the multi-well cassette is returned to fluid handling system 16 by automated cassette handling system 18. At step 82, the T₁ sample is stained by automated pipette handling system 24. At the same time, at step 84, samples in AT wells also are stained. Thereafter, at step 86 the T₁ sample is enumerated (FIGS. 12A and 12B) and the growth ratio after incubation, i.e., ratio of T₁ to T₀ cells, is determined at step 88.

Enumeration (80, 86), determination (88) and assessment of the T₁/T₀ cell growth ratio (89) are important steps to allow quantitative discrimination between pathogenic cells/bacteria of interest and contaminant cells/bacteria. It has been determined by the Applicant that pathogenic bacteria exhibit different growth rates as compared to non-pathogenic, contaminant bacteria and that these differences in growth rate may be used to discriminate quantitatively between cells of clinical interest and contaminant cells, without reliance on more subjective, qualitative measures such as turbidity of plated samples. For example, it has been determined that pathogenic cells in human urine exhibit a growth rate that is approximately 2.25±1 greater than the growth rate of contaminant cells when cultured over short culture times in the range of approximately 2.5 hours. It may be possible in certain circumstances to state the growth rate difference more specifically as 2.25±0.5. Thus in one embodiment, if the T₁ to T₀ cell growth ratio is determined to be between about 1.25 and 3.25 (i.e., about 125% to about 325%) the sample may be assessed (step 89) as a positive for pathogenic bacteria (89A).

In another embodiment, the system may be programmed to convert the relative growth between T0 and T1 to an integer representing bacterial population expansion. In such an embodiment, the derived growth integer from T0 baseline to T1 control growth is compared to the known growth integers of a known library of pathogens represented in the disease state being tested. Representative disease states may include, but are not limited to, pathogens associated with urinary tract infections, pathogens associated with blood stream infections (bacteremia/sepsis), pathogens associated with meningitis or other neurologic infections. Alternatively or additionally, the derived growth integer is compared to the known growth integers of a known library of possible bacterial contaminants represented in the disease state being assessed, such as, but not limited to normal urogenital flora associated with suspected urinary tract infections or possible skin contaminant associated with blood sampling in suspected bacteremia samples. Known libraries of pathogens and contaminants may be stored in fluid library 42 in memory 36.

Depending on the clinical objective, for example if simply determining existence of a urinary tract infection is the goal, then the positive result may be the stopping point and the result reported to the appropriate health care provider or patient. However, embodiments of the present invention also provide for rapid assessment of antibiotic resistance/susceptibility prediction if such information is desired. If the result of the assessment in step 89 is positive, enumeration of the samples placed in the AT wells may proceed. Because the samples were distributed to the AT wells at the same time as the T₀ and T₁ wells, the samples in the AT wells were cultured also during incubation step 78 and thus may be immediately enumerated without additional culture time. At steps 90, 92, et seq., samples from AT wells 1-n are enumerated to determine an antibiotic prediction profile or for use as information in determining antibiotic susceptibility based on comparison with the T₁ sample. For these comparisons, the T1 enumeration provides a baseline against which the AT well enumeration will be compared. Resistance prediction may be based on growth rate thresholds as may be established for specific clinical indications and/or drugs and antibiotics. Note that once again, by using flow cytometer enumeration and comparing the ratio of, e.g., ATₙ/T₁, a quantitative measurement of the antibiotic/drug effectiveness may be determined.

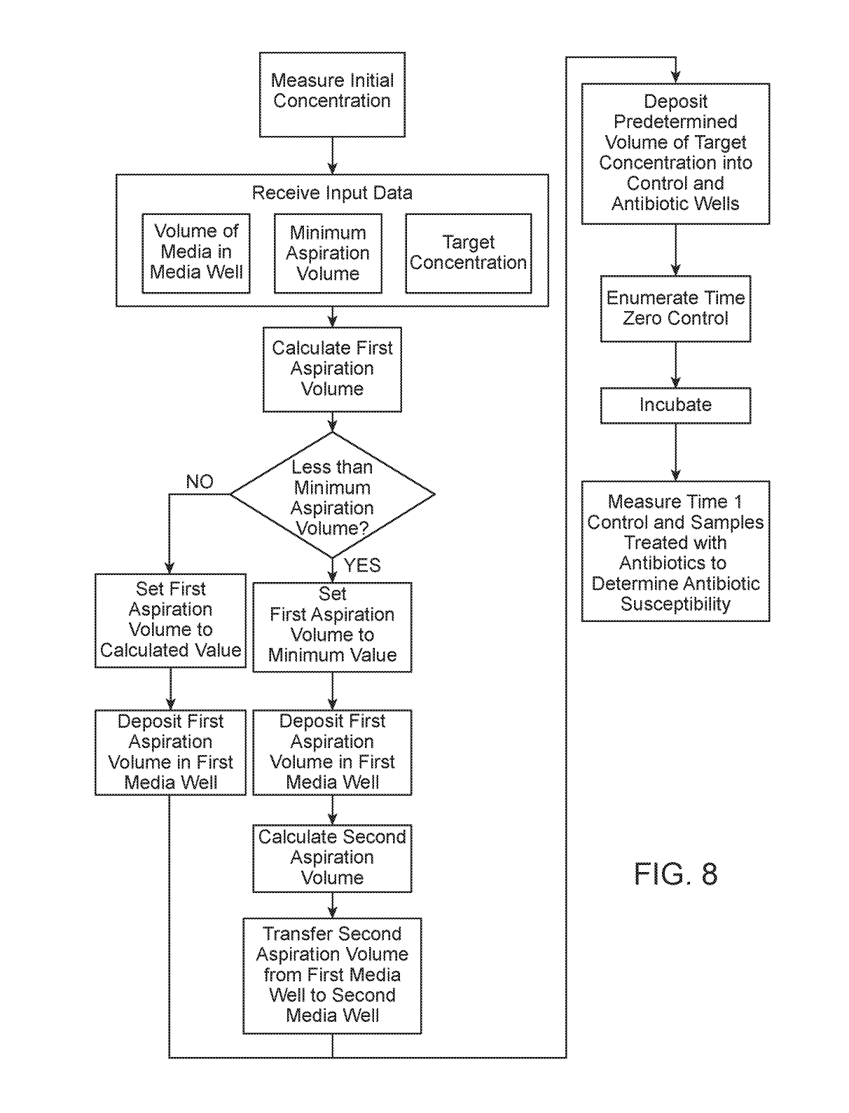

In other embodiments, systems also may be configured to automatically adjust the initial sample concentration to a predetermined concentration for subsequent testing. As shown in FIG. 8, the initial sample, after dyeing and, in some cases, the addition of compensation beads, may be measured by flow cytometer 22 to obtain an initial concentration of bacteria and an initial determination of infection. The illustrated flow cytometer and fluid handling system software modules 46, 50 may be configured to obtain fluid and flow cytometer specific parameters from fluid library 42 for determining whether the initial measurements indicate an infection. With the initial concentration of bacteria determined, the fluid handling system software may automatically calculate a required concentration adjustment for further testing. The software may also determine, based on the volume of growth media deposited in the media wells, an amount of the fluid sample to be deposited in a first media well to arrive at the required concentration for further testing. In some examples, a minimum amount of the fluid sample, e.g., >1 microliter may be required to be aspirated to ensure accurate volumes of fluid transport by the system. As shown in FIG. 8, depending on the initial concentration of bacteria, required dilution, minimum aspiration volume, and volume of growth media in the media wells, a multi-step dilution process may be required to arrive at the target concentration.

For example, various antimicrobial efficacy testing methods may require a standard concentration of bacteria, e.g., a predetermined bacterial concentration of 1×10⁴ bacteria/ml. If initial testing of a clinical sample indicates a higher concentration, e.g., if the flow cytometer enumerates an initial sample at 1×10⁷ bacteria/ml, the system may automatically adjust the concentration for subsequent testing. In one example, 1 microliter of the sample may be aspirated by the fluid handling system and deposited into 1000 microliters of media in a first one of the media wells to arrive at the target concentration of 1×10⁴. In another example, the initial concentration may be greater than 1×10⁷ bacteria/ml, and/or the minimum aspiration volume may be greater than 1 microliter, and/or the target concentration may be lower, etc. such that a second dilution step is required. The fluid handling system may be configured to determine a second amount of fluid to be aspirated from the first media well containing media and the first amount of the fluid sample for deposit in a second media well to arrive at the target concentration, e.g., 1×10⁴ bacteria/ml.

After automatically creating a target concentration of fluid that contains one or more of (1) a portion of the initial fluid sample (2) dye (3) compensation beads and (4) dilution media, e.g., growth media, the fluid handling system software module may include instructions for causing the fluid handling system to automatically deposit predetermined amounts of the target concentration in one or more of the antibiotic wells (FIG. 2) and in the two control wells. The fluid handling system may then automatically aspirate a predetermined amount of fluid from one of the control wells (hereinafter the time zero control well) and transport the control to flow cytometer 22 for obtaining a time zero bacteria count measurement. After transmitting the time zero control sample to the flow cytometer, the cassette may then be transported, e.g., by hand or with the cassette transporter, to the incubator for incubation for a period of time at a controlled temperature, e.g., at physiologic temperature, such as 35-37° C. By utilizing a flow cytometer for enumerating bacteria count, the required incubation time is significantly reduced as compared to current bacteria susceptibility testing, e.g., an incubation time of approximately 1-3 hours as compared to required prior art times of 1-2 days. As will be appreciated by a person having ordinary skill in the art, such a reduction in time is invaluable, allowing comprehensive susceptibility testing prior to treatment, thereby enabling a targeted antibiotic treatment and avoiding the chronic over-prescription of large numbers of antibiotics as is currently done in the art.

After incubation, the cassette may be transported back to the fluid handling system and the fluid mixture in the second control well and in the antibiotic wells may be automatically analyzed by separately transmitting a controlled volume of each to the flow cytometer for analysis. Between each flow cytometer reading, the system may also be configured to operate the wash system, which may include a fluid reservoir of washing fluid, e.g., a disinfecting fluid, that may be used for flushing the fluid handling system, and, in some examples, the flow cytometer.

The system may utilize the time zero and subsequent control measurement to determine a uninhibited bacteria growth rate. And the system can also compare the difference in bacteria count between the time zero control and the subsequent measurement of each antibiotic well to determine if the bacteria in the given sample is susceptible to a given antibiotic, including determining if a given antibiotic is static (statistically same enumeration as time zero control) or cidal (a statistically lower enumeration than the time zero control).

### Intra-Assay Compensation Particles

Described herein are methods for compensating for inaccurate enumeration of target populations by flow cytometers due to not counting or not capturing every event of interest. Such inaccurate enumeration may occur due to limitations in the sensors and/or other data acquisition components and software of the system. For example, a flow cytometer may not count every particle of interest when faced with samples containing excess particles, which causes underestimation of the accurate enumeration of target populations.

Micro-beads are commonly used for flow cytometer calibration before samples are analyzed to determine if the instrument is operating properly. In embodiments described herein, micro-beads are incorporated directly into a sample, i.e., as intra-assay compensation particles, for use in determining enumeration accuracy and compensating for undercounting when, for example, the data acquisition threshold of the instrument has been exceeded. In one example, a known number of compensation particles, such as fluorescent micro-beads, are added to a sample in order to quantify an inaccuracy of the flow cytometer reading. Exemplary compensation particles may have unique scatter and fluorescent characteristics, where these characteristics are distinct enough from the target population that they can be easily distinguished and enumerated. In one example, a concentration of between about 50 to 300 compensation particles/μl are added to a sample prior to enumeration. More specifically the concentration of compensation particles in a sample may be about 200 compensation particles/O. In other examples, other concentrations may be used.

In situations where data acquisition has met or exceeded a threshold for enumerative accuracy, the instrument's enumeration of the compensation particle population will also be inaccurate, providing a particle population count that is below an expected value based on the known compensation particle concentration added to the sample prior to enumeration. The difference between the number of expected events (based on the known number of micro-beads added to the sample) and the enumerated events can be used to adjust the reported enumeration of a target population to more accurately represent the actual value present in a sample. For example, a scaling factor can be determined based on the ratio of the measured number of compensation particles to the expected number. The scaling factor can then be applied to the measured number of the population of interest, such as bacteria. In one example, a direct 1:1 linear scaling factor is applied to the measured value that assumes a 1:1 relationship between the percent inaccuracy in the compensation particle measurement to the percent inaccuracy in the particle of interest measurement. For example, if only 80% of a known number of compensation particles are detected, the number of events of interest may also be only 80% of an actual number. The measured number may, therefore, be increased by 20%. In other examples, an empirically-based multiplier may be applied to the scaling factor that assumes a linear relationship other than 1:1. In yet other examples, a non-linear scaling factor may be applied.

Such a scaling factor may be used to develop a more accurate enumeration of bacteria in a sample, for example, to determine whether there is an infection. In some examples, the concentration of the sample may then need to be reduced for subsequent testing and analysis, such as for antimicrobial efficacy testing. In one example, there may be a target concentration for antimicrobial testing, such as approximately 1×10⁵ bacteria/ml to approximately 5×10⁵ bacteria/ml; or approximately 1×10⁴ bacteria/ml to approximately 5×10⁴ bacteria/ml; or approximately 1×10³ bacteria/ml to approximately 5×10³ bacteria/ml, or an target concentration falling within a specified band of an overall concentration in the range of 1×10³ bacteria/ml to about 5×105 bacteria/ml, among others. A method of determining antimicrobial efficacy for a sample may include an initial test with a flow cytometer to make a determination of infection and to determine the concentration of bacteria in the sample. As described above, compensation particles may be used to determine whether flow cytometer system data acquisition has been exceeded. If so, a scaling factor may be determined as described above and applied to the measured number of bacteria to calculate an actual bacterial concentration in the initial sample. The actual concentration may then be used to determine the dilution process required to arrive at the target concentration required for subsequent testing.

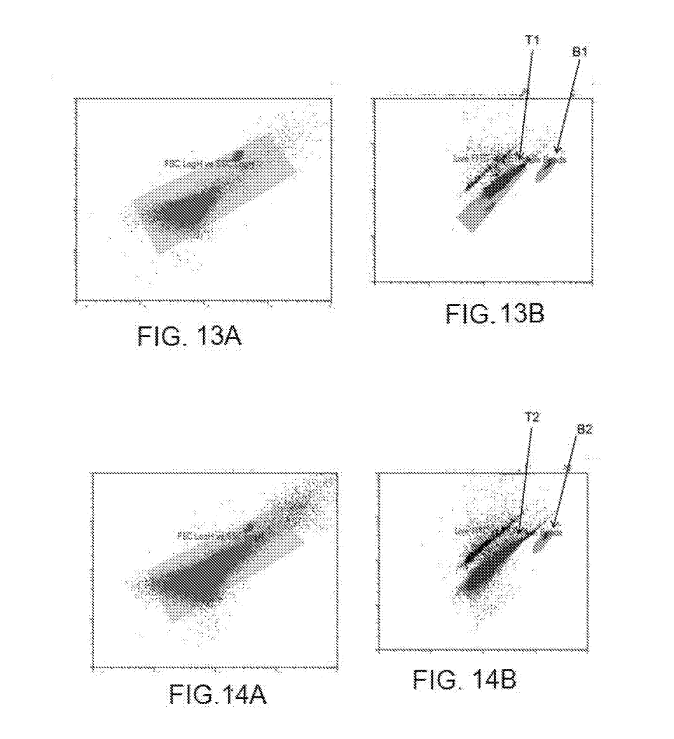

As an example, FIGS. 13A-B are cytograms representing a clinical specimen that has been diluted. FIG. 13A represents events within a sample based on forward and side scatter with the shaded “window” representing a gate developed to identify particles of interest, e.g., one or more species of bacteria. The image on the right represents the fluorescence gates for the sample, including the compensation particles present in the sample. As is known in the art, the fluorescence enumeration shown in FIG. 13B only takes place on events that fall within the gate shown in the FIG. 13A scatter plot.

Expected compensation values B1 are 84/ul, whereas the known actual value is 82/ul. In one example, this may be considered sufficiently accurate, indicating the data acquisition system capability has not been exceeded by determining whether the difference between measured and actual is within a known statistical accuracy of the instrument. The expected value of the target population T₁, therefore, can be considered accurate.

FIGS. 14A-B illustrate another example, where the same number of compensation particles (82/ul) were used in a different sample. A comparison of FIGS. 13A and 14A show there are significantly more particles detected in the sample shown in FIG. 14A. In the second example, the compensation particle population enumerated at 69/ul, which is less than the known population. In the illustrated example, the difference is greater than a difference due to known statistical variation in the instrument during normal operation, indicating the data acquisition system capability has been exceeded. This indicates the enumeration of a target population, such as target population T₂ (FIG. 14B) may also be lower than actual. In one example, a scaling factor may be applied to any target population enumeration, such as the enumeration of population T₂ to account for the error. In one example, the factor may be determined based on a ratio of measured to known enumeration of compensation particles. In the example shown in FIG. 14B, a factor of approximately 1.2 may be applied to the enumerated target population. With the factor applied, a more accurate bacterial concentration is determined, which can be used for determining the extent of dilution required to prepare a target concentration for subsequent antimicrobial efficacy testing.

It is to be noted that any one or more of the aspects and embodiments described herein may be conveniently implemented using one or more machines (e.g., one or more computing devices) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

FIG. 15 shows a diagrammatic representation of one alternative embodiment of processing and control unit 12 in the exemplary form of a computer system 1500 within which a set of instructions for causing a control system, such as hardware system 15 of FIGS. 1 and 2, to perform any one or more of the aspects and/or methodologies of the present disclosure may be executed. It is also contemplated that multiple computing devices may be utilized to implement a specially configured set of instructions for causing one or more of the devices, such as flow cytometer 22, to perform any one or more of the aspects and/or methodologies of the present disclosure. Computer system 1500 includes a processor 1504 and a memory 1508 that communicate with each other, and with other components, via a bus 1512. Bus 1512 may include any of several types of bus structures including, but not limited to, a memory bus, a memory controller, a peripheral bus, a local bus, and any combinations thereof, using any of a variety of bus architectures.

Memory 1508 may include various components (e.g., machine-readable media) including, but not limited to, a random access memory component, a read only component, and any combinations thereof. In one example, a basic input/output system 1516 (BIOS), including basic routines that help to transfer information between elements within computer system 1500, such as during start-up, may be stored in memory 1508. Memory 1508 may also include (e.g., stored on one or more machine-readable media) instructions (e.g., software) 1520 embodying any one or more of the aspects and/or methodologies of the present disclosure. In another example, memory 1508 may further include any number of program modules including, but not limited to, an operating system, one or more application programs, other program modules, program data, and any combinations thereof.

Computer system 1500 may also include a storage device 1524. Examples of a storage device (e.g., storage device 1524) include, but are not limited to, a hard disk drive, a magnetic disk drive, an optical disc drive in combination with an optical medium, a solid-state memory device, and any combinations thereof. Storage device 1524 may be connected to bus 1512 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1524 (or one or more components thereof) may be removably interfaced with computer system 1500 (e.g., via an external port connector (not shown)). Particularly, storage device 1524 and an associated machine-readable medium 1528 may provide nonvolatile and/or volatile storage of machine-readable instructions, data structures, program modules, and/or other data for computer system 1500. In one example, software 1520 may reside, completely or partially, within machine-readable medium 1528. In another example, software 1520 may reside, completely or partially, within processor 1504.

Computer system 1500 may also include an input device 1532. In one example, a user of computer system 1500 may enter commands and/or other information into computer system 1500 via input device 1532. Examples of an input device 1532 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1532 may be interfaced to bus 1512 via any of a variety of interfaces (not shown) including, but not limited to, a serial interface, a parallel interface, a game port, a USB interface, a FIREWIRE interface, a direct interface to bus 1512, and any combinations thereof. Input device 1532 may include a touch screen interface that may be a part of or separate from display 1536, discussed further below. Input device 1532 may be utilized as a user selection device for selecting one or more graphical representations in a graphical interface as described above.

A user may also input commands and/or other information to computer system 1500 via storage device 1524 (e.g., a removable disk drive, a flash drive, etc.) and/or network interface device 1540. A network interface device, such as network interface device 1540, may be utilized for connecting computer system 1500 to one or more of a variety of networks, such as network 1544, and one or more remote devices 1548 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1544, may employ a wired and/or a wireless mode of communication. In general, any network topology may be used. Information (e.g., data, software 1520, etc.) may be communicated to and/or from computer system 1500 via network interface device 1540.

Computer system 1500 may further include a video display adapter 1552 for communicating a displayable image to a display device, such as display device 1536. Examples of a display device include, but are not limited to, a liquid crystal display (LCD), a cathode ray tube (CRT), a plasma display, a light emitting diode (LED) display, and any combinations thereof. Display adapter 1552 and display device 1536 may be utilized in combination with processor 1504 to provide graphical representations of aspects of the present disclosure. In addition to a display device, computer system 1500 may include one or more other peripheral output devices including, but not limited to, an audio speaker, a printer, and any combinations thereof. Such peripheral output devices may be connected to bus 1512 via a peripheral interface 1556. Examples of a peripheral interface include, but are not limited to, a serial port, a USB connection, a FIREWIRE connection, a parallel connection, and any combinations thereof.

The foregoing has been a detailed description of illustrative embodiments of the invention. It is noted that in the present specification and claims appended hereto, conjunctive language such as is used in the phrases “at least one of X, Y and Z” and “one or more of X, Y, and Z,” unless specifically stated or indicated otherwise, shall be taken to mean that each item in the conjunctive list can be present in any number exclusive of every other item in the list or in any number in combination with any or all other item(s) in the conjunctive list, each of which may also be present in any number. Applying this general rule, the conjunctive phrases in the foregoing examples in which the conjunctive list consists of X, Y, and Z shall each encompass: one or more of X; one or more of Y; one or more of Z; one or more of X and one or more of Y; one or more of Y and one or more of Z; one or more of X and one or more of Z; and one or more of X, one or more of Y and one or more of Z.

Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this invention.

Further alternative exemplary embodiments of the present disclosure are described in the paragraphs below.

In one example, a method of using a flow cytometer for testing a sample of a body fluid for the presence of bacteria, which includes dividing the sample into at least two batches including a Time 0 control (T₀ control) and a Time 1 control (T₁ control); testing the T₀ control at time T₀ with the flow cytometer to obtain T₀ enumerative baseline bacterial values relating to measured characteristics of the T₀ sample; testing the T₁ control at time with the flow cytometer to obtain T₀ enumerative baseline bacterial values relating to measured characteristics of the T₀ sample; comparing the T₁ control values to the T₀ baseline values to determine the growth integer of samples containing bacteria. The T₀ baseline values and the T₁ control values may include live cell events in a bacteria-specific region of interest (ROI), the comparing step including comparing the live cell events at T₀ and T₁ and determining whether live bacteria is present when there is a statistically significant increase in the number of live cell events at T₁ as compared to T₀.

The enumerative accuracy of the bacteria-specific region at all testing times is improved by utilizing a test-enumerative compensator (TEC) particle, such as a bead with flow cytometric scatter and fluorescence characteristics similar, yet slightly altered from the target population, within the test run with known expected values with the aim of compensating data acquisition deficiencies during the test run. A unique ROI separate from the live bacteria ROI may be created to enumerate the TEC during every analysis run.

Additionally, at least two batches further include n AT samples, each one of the n AT samples being treated by a different one of n different antibiotics, wherein n is an integer greater than zero, the method further comprising: testing each of the n AT samples at time T₁ with the flow cytometer to obtain n AB sample values; comparing the T₀ control live cell events in the ROI to each of the n T₁ sample live cell events in the ROI to determine the susceptibility or resistance of detected bacteria to the n different antibiotics. As desired, all AT samples tested for bacterial enumeration values are may be compensated using TEC compensator particles according to the method described above.

The antibiotic concentrations being tested generally may represent the clinically accepted lower interpretive breakpoint defined by Clinical Laboratory Standards Institute for each individual antibiotic being tested. Further, the antibiotic concentrations being tested may represent the clinically accepted range of antibiotic concentrations used to define the minimum inhibitory concentration of each antibiotic defined by Clinical Laboratory Standards Institute for each individual antibiotic being tested.

Additional embodiments may involve comparing the T₁ control values and the n T₁ sample values to detect the presence of multiple sub-populations of bacteria due to the sub-populations having a differing response to any one of the n antibiotics. Body fluids may be selected from the group consisting of at least urine, blood, pleural fluid, synovial fluid or cerebral spinal fluid. The flow cytometer system includes software containing separate body-fluid-specific data sets for each of the urine, blood, or cerebral spinal fluid that accounts for: known matrix noise and provides statistical confidence information specific to the body fluid type; pre-defined growth integers for pathogens associated with pathological bacterial infections; and pre-defined growth integers for possible contaminants associated with normal sampling.

When the body fluid from which the sample is taken is blood, the presence of bacteria and antibiotic susceptibility can be determined after only about 2 to 12 hours of incubating (as opposed to the 18-22 hours of incubating time currently required to allow bacteria to grow enough to be detected using current techniques).

Using automated fluid handling techniques, the original clinical sample being divided may divided by an automatic fluid handling system between the clinical sample the T₀ sample and the T₁ sample. Further, all relevant staining reagents used for bacterial determinations are added using an automated fluid handling system that aspirates, deposits, and mixes the reagents and samples. Additionally, all n AT samples may created from the clinical sample using automated fluid handling.

In further embodiments, a non-transient computer readable medium or computer program product may be provided with or store a flow cytometer software analysis program and algorithm for automatically performing testing, enumerative evaluations and comparisons as described hereinabove, to automatically detects the presence of live bacteria of interest and to automatically discriminate between pathogenic and non-pathogenic, contaminant bacteria. In one example a flow cytometer software analysis program may also include stored instruction to execute one or more of the following functions in an flow cytometer-based sequential analysis system: automatically determine the quantitative effect of antibiotics tested, automatically determine the relative effect of antibiotics tested and report via a graphical user interface whether the bacteria is susceptible to the antibiotic being tested, automatically determine the relative effect of antibiotics tested and report through the graphical user interface whether the bacteria is resistant to the antibiotic being tested, automatically compensate the enumerative values of bacteria or other particles of interest using TEC particles placed in the sample by the automated fluid handling system prior to testing and/or provide information on whether more than one sub-population of bacteria is present in the sample indicating likelihood of co-infection based on population densities and statistical evaluation of events within the live bacteria ROI.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A method of using a flow cytometer in an automated fluid handling system for testing a clinical sample of a body fluid for the presence of bacteria, and optionally determining sample response to at least one antibiotic, comprising:
distributing a portion of the sample to at least a first test well using the automated fluid handling system;
testing the sample portion from the first well with a flow cytometer to determine a total bacteria count;
adjusting dilution of the sample with growth media to a predetermined concentration based on the total bacteria count;
dividing the dilution-adjusted sample into at least wells including a time 0 baseline (T₀ baseline) well and a time 1 control (T₁ control) well;
testing the sample in the T₀ baseline well at time T₀ with the flow cytometer to obtain T₀ enumerative baseline bacterial values relating to measured characteristics of the sample in the T₀ well;
culturing the sample in the T₁ control well from time 0 to time 1;
testing the T₁ control batch at time 1 with the flow cytometer to obtain T₁ enumerative control bacterial values relating to measured characteristics of the T₁ sample; and
comparing the T₁ control values to the T₀ baseline values to determine a growth ratio of samples containing bacteria.

2. The method according to claim 1, further comprising, at or approximate to time 0:
inoculating at least one antibiotic test (AT) well each with an antibiotic of interest;
distributing dilution-adjusted portions of the sample to at least one AT well using the automated fluid handling system;
testing the sample portion from the first well with a flow cytometer to determine AT enumerative bacterial values relating to measured characteristics of the sample in the at least one AT well; and
comparing the AT enumerative values to the T1 control values to determine a response of the sample to the antibiotics of interest.

3. The method according to claim 1, wherein the T₀ baseline values and the T₁ control values include cell events of interest in a bacteria-specific region of interest (ROI), the comparing step including comparing the cell events of interest at T₀ and T₁ and determining whether cells of interest are present when there is a statistically significant increase in the number of cell of interest events at T₁ as compared to T₀.

4. The method according to claim 1, wherein the cells of interest are pathogenic bacteria.

5. The method according to claim 1, further comprising:
converting a relative growth between T₀ and T₁ to a growth integer representing bacterial population expansion;
comparing the growth integer from T₀ baseline and T₁ control to at least one known growth integer from a known library of pathogens represented in a disease state being tested; and
determining the type of pathogen present in the sample based on said comparing.

6. The method according to claim 1, further comprising:
converting a relative growth between T₀ and T₁ to a growth integer representing bacterial population expansion;
comparing the growth integer from T₀ control and T₁ to known growth integers of a known library of possible bacterial contaminants represented in a disease state being assessed; and
determining the type of bacterial contaminants present in the sample based on said comparing.

7. The method according to claim 3, wherein at least two wells further includes n AT samples, each one of the n AT samples being treated by a different one of n different antibiotics, wherein n is an integer greater than zero, the method further comprising:
testing each of the n AT samples at time T₁ with the flow cytometer to obtain n AT sample values; and
comparing the T₀ baseline events in the ROI to each of the n T₁ sample events in the ROI to determine the susceptibility or resistance of detected bacteria to the n different antibiotics.

8. The method according to claim 7, further comprising comparing the T₁ control values and the n AT sample values to detect the presence of multiple sub-populations of bacteria due to the sub-populations having a differing response to any one of the n antibiotics.

9. The method according claim 1, wherein the body fluid is selected from the group consisting of urine, blood, pleural fluid, synovial fluid or cerebral spinal fluid.

10. The method according to claim 9, wherein the flow cytometer is controlled by a processor executing instructions stored in a memory, said memory further containing separate body-fluid-specific data sets for each of the urine, blood, or cerebral spinal fluid wherein each said data set accounts for:
a. known matrix noise and provides statistical confidence information specific to the body fluid type,
b. pre-defined growth integers for pathogens associated with pathological bacterial infections, and
c. pre-defined growth integers for possible contaminants associated with normal sampling.

11. The method according to claim 1, wherein the sample is divided by an automatic fluid handling system between an clinical sample, the T₀ sample and the T₁ sample.

12. The method according to claim 1, wherein relevant staining reagents used for bacterial determinations are added using an automated fluid handling system that aspirates, deposits, and mixes the reagents and samples.

13. The method according to claim 8, where all n AT samples are created from the clinical sample using automated fluid handling.

14. The method according to claim 1, further comprising:
including a known concentration of a test-enumerative compensator (TEC) particles in the sample, said TEC particles having known flow cytometric scatter and fluorescence characteristics;
enumerating the TEC particles with the sample testing by the flow cytometer;
determining a compensator factor based on the enumerated TEC particle value as compared to the known TEC particle concentration in the sample tested; and
adjusting the sample test enumeration value by said compensator factor.

15. The method according to claim 14, wherein said enumerating TEC particles is included with each flow cytometer sample test.

16. The method according to claim 14, wherein said determining comprises applying a unique TEC particle ROI separate from the bacteria ROI for enumerating the TEC particles.

17. A method of using a flow cytometer for testing a sample of a body fluid for the presence of bacteria, and optionally determining sample response to at least one antibiotic, comprising:
adjusting dilution of the sample to a predetermined concentration;
dividing the diluted sample into at least two batches including a time 0 baseline (T₀ baseline) batch and a time 1 control (T₁ control) batch;
testing the T₀ baseline batch at time T₀ with the flow cytometer to obtain T₀ enumerative baseline bacterial values relating to measured characteristics of the T₀ batch;
culturing the T1 control batch in growth media from time 0 to time 1;
testing the T₁ control batch at time 1 with the flow cytometer to obtain T₁ enumerative control bacterial values relating to measured characteristics of the T₁ sample; and
comparing the T₁ control values to the T₀ baseline values to determine a growth ratio of samples containing bacteria.

18. The method according to claim 17, wherein the T₀ baseline values and the T₁ control values include cell events of interest in a bacteria-specific region of interest (ROI), the comparing step including comparing the cell events of interest at T₀ and T₁ and determining whether cells of interest are present when there is a statistically significant increase in the number of cell of interest events at T₁ as compared to T₀.

19. The method according to claim 17, wherein at least two wells further includes n AT samples, each one of the n AT samples being treated by a different one of n different antibiotics, wherein n is an integer greater than zero, the method further comprising:
testing each of the n AT samples at time T₁ with the flow cytometer to obtain n AT sample values; and
comparing the T₀ baseline cell events in the ROI to each of the n T₁ sample cell events in the ROI to determine the susceptibility or resistance of detected bacteria to the n different antibiotics.

20. The method according to claim 18, where in the statistically significant increase is an increase of about 125% to about 325%.

21. A method of compensating for inaccuracies in flow cytometer enumeration of particles of interest in fluid samples, comprising:
including a known concentration of a test-enumerative compensator (TEC) particles in the sample to be enumerated, said TEC particles having known flow cytometric scatter and fluorescence characteristics;
enumerating the TEC particles with the sample enumeration by the flow cytometer;
determining a compensator factor based on the enumerated TEC particle value as compared to the known TEC particle concentration in the sample tested; and
adjusting the sample test enumeration value by said compensator factor.

22. The method according to claim 21, wherein said determining comprises applying a unique TEC particle ROI separate from the particle of interest ROI for enumerating the TEC particles.

23. The method according to claim 21, wherein the known flow cytometric scatter and fluorescence characteristics of the TEC particles are similar to the corresponding characteristics of the particles of interest.

24. A system for automated testing a sample of a body fluid for the presence of bacteria, and optionally determining sample response to at least one antibiotic, the system comprising:
fluid handling device including an automated pipetting system for distributing fluid samples among wells of a well plate;
incubator configured to culture samples in well plates received from the fluid handling device;
plate transport device configured to deliver well plates containing samples to the incubator from the fluid handling device and return well plates from the incubator to the fluid handling device;
flow cytometer configured to enumerate cell counts in samples provided by the fluid handling system;
processor and memory, the processor configured execute instructions stored in the memory to control the system in accordance with said instructions, wherein the stored instructions cause the system to —
distribute a portion of the sample to at least a first test well;
enumerate the sample portion from the at least first test well to determine a total bacteria count;
adjust dilution of the sample with growth media to a predetermined concentration based on the total bacteria count;
divide the dilution-adjusted sample into at least wells including a time 0 baseline (T₀ baseline) well and a time 1 control (T₁ control) well;
enumerate the sample in the T₀ baseline well at time T₀ to obtain T₀ enumerative baseline bacterial values relating to measured characteristics of the sample in the T₀ well;
deliver the sample to the incubator;
culture the sample in the T1 control well from time 0 to time 1;
return the sample to the fluid handling device after culturing;
enumerate the T₁ control batch at time 1 with the flow cytometer to obtain T₁ enumerative control bacterial values relating to measured characteristics of the T₁ sample; and
compare the T₁ control values to the T₀ baseline values to determine a growth ratio of samples containing bacteria; and
a graphical user interface communicating with at least the processor allowing user interaction with the system.

25. The system according to claim 25, wherein said instructions stored in memory further cause the system, at or approximate to time 0:
distribute dilution-adjusted portions of the sample to at least to at least one AT well inoculated with an antibiotic of interest;
enumerate the sample portion from the first well with a flow cytometer to determine AT enumerative bacterial values relating to measured characteristics of the sample in the at least one AT well; and
compare the AT enumerative values to the T₁ control values to determine a response of the sample to the antibiotics of interest.
